(19) United States
(12) Patent Application Publication
(10) Pub. No.: US 2022/0151647 A1
(43) Pub. Date: May 19, 2022

(54) CATHETERS HAVING SHAPED DISTAL PORTIONS, AND ASSOCIATED SYSTEMS AND METHODS

(71) Applicant: Inari Medical, Inc., Irvine, CA (US)

(72) Inventors: Edward Enriquez Dolendo, Mission Viejo, CA (US); Thomas Robert Brodt, Long Beach, CA (US); Thomas M. Tu, Louisville, KY (US)

(21) Appl. No.: 17/529,018

(22) Filed: Nov. 17, 2021

Related U.S. Application Data
(60) Provisional application No. 63/115,512, filed on Nov. 18, 2020.

(51) Int. Cl. A61B 17/22; A61M 25/09; A61M 25/00

(52) U.S. Cl. CPC A61B 17/22; A61M 25/09; A61M 25/0023; A61B 2017/22079; A61M 2025/09091; A61M 2025/0025; A61M 25/005

(57) ABSTRACT

Disclosed herein are aspiration guide catheters having a pre-shaped distal portion configured to deflect away from a longitudinal axis of the catheter, and associated systems and methods. In some embodiments, an aspiration guide catheter includes an inner liner defining a lumen and having a proximal region and a distal region. The catheter further includes a braid of wires over the inner liner, and a wire coiled around the braid over at least a portion of the distal region of the inner liner. At least a portion of the braid over the distal region of the inner liner is configured to deflect away from a longitudinal axis of the proximal region. The catheter can further include an outer sheath over the braid, the wire, and the inner liner.

### CROSS-REFERENCE TO RELATED APPLICATION

This application claims the benefit of U.S. Provisional Patent Application No. 63/115,512, filed Nov. 18, 2020, and titled “CATHETERS HAVING SHAPED DISTAL PORTIONS, AND ASSOCIATED SYSTEMS AND METHODS,” which is incorporated herein by reference in its entirety.

### TECHNICAL FIELD

The present technology generally relates to catheters having shaped (e.g., pre-shaped) distal portions, such as a distal curved portion, to facilitate positioning of the catheter in hard-to-reach regions of the vasculature of a patient.

### BACKGROUND

Thromboembolic events are characterized by an occlusion of a blood vessel. Thromboembolic disorders, such as stroke, pulmonary embolism, heart attack, peripheral thrombosis, atherosclerosis, and the like, affect many people. These disorders are a major cause of morbidity and mortality.

When an artery is occluded by a clot, tissue ischemia develops. The ischemia will progress to tissue infarction if the occlusion persists. Infarction does not develop or is greatly limited if the flow of blood is reestablished rapidly. Failure to reestablish blood flow can lead to the loss of limb, angina pectoris, myocardial infarction, stroke, or even death.

In the venous circulation, occlusive material can also cause serious harm. Blood clots can develop in the large veins of the legs and pelvis, a common condition known as deep venous thrombosis (DVT). DVT arises most commonly when there is a propensity for stagnated blood (e.g, long distance air travel, immobility, etc.) and clotting (e.g., cancer, recent surgery, such as orthopedic surgery, etc.). DVT causes harm by: (1) obstructing drainage of venous blood from the legs leading to swelling, ulcers, pain, and infection, and (2) serving as a reservoir for blood clots to travel to other parts of the body including the heart, lungs, brain (stroke), abdominal organs, and/or extremities.

In the pulmonary circulation, the undesirable material can cause harm by obstructing pulmonary arteries—a condition known as pulmonary embolism. If the obstruction is upstream, in the main or large branch pulmonary arteries, it can severely compromise total blood flow within the lungs, and therefore the entire body, and result in low blood pressure and shock. If the obstruction is downstream, in large to medium pulmonary artery branches, it can prevent a significant portion of the lung from participating in the exchange of gases to the blood resulting in low blood oxygen and buildup of blood carbon dioxide.

There are many existing techniques to reestablish blood flow through an occluded vessel. One common surgical technique, an embolectomy, involves incising a blood vessel and introducing a balloon-tipped device (such as the Fogarty catheter) to the location of the occlusion. The balloon is then inflated at a point beyond the clot and used to translate the obstructing material back to the point of incision. The obstructing material is then removed by the surgeon. Although such surgical techniques have been useful, exposing a patient to surgery may be traumatic and best avoided when possible. Additionally, the use of a Fogarty catheter may be problematic due to the possible risk of damaging the interior lining of the vessel as the catheter is being withdrawn.

Percutaneous methods are also utilized for reestablishing blood flow. A common percutaneous technique is referred to as balloon angioplasty where a balloon-tipped catheter is introduced to a blood vessel (e.g., typically through an introducing catheter). The balloon-tipped catheter is then advanced to the point of the occlusion and inflated to dilate the stenosis. Balloon angioplasty is appropriate for treating vessel stenosis, but it is generally not effective for treating acute thromboembolisms as none of the occlusive material is removed and the vessel will re-stenos after dilation. Another percutaneous technique involves placing a catheter near the clot and infusing streptokinase, urokinase, or other thrombolytic agents to dissolve the clot. Unfortunately, thrombolysis typically takes hours to days to be successful. Additionally, thrombolytic agents can cause hemorrhage and in many patients the agents cannot be used at all.

Various devices exist for performing a thrombectomy or removing other foreign material. However, such devices have been found to have structures which are either highly complex, cause trauma to the treatment vessel, or lack sufficient retaining structure and thus cannot be appropriately fixed against the vessel to perform adequately. Furthermore, many of the devices have highly complex structures that lead to manufacturing and quality control difficulties as well as delivery issues when passing through tortuous or small diameter catheters. Less complex devices may allow the user to pull through the clot, particularly with inexperienced users, and such devices may not completely capture and/or collect all the clot material.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a partially schematic side view of a clot treatment system including a catheter in accordance with embodiments of the present technology.

FIG. 2 is an enlarged, partial cut-away side view of a portion of a proximal region of the catheter of FIG. 1 in accordance with embodiments of the present technology.

FIG. 3A is an enlarged, partial cut-away side view, and FIG. 3B is an enlarged, partial cut-away perspective view, of a portion of a distal region of the catheter of FIG. 1 in accordance with embodiments of the present technology.

FIG. 3C is an enlarged cross-sectional view of the distal region of the catheter of FIG. 1 taken along the line 3C-3C in FIG. 3B in accordance with embodiments of the present technology.

FIGS. 4A and 4B are side views of the distal portion of the catheter of FIG. 1 during a procedure for removing clot material from within a blood vessel of a patient in accordance with embodiments of the present technology.

FIGS. 5A and 5B are side views of the distal portion of the catheter of FIG. 1 during a procedure for removing clot material from within a blood vessel of a patient in accordance with additional embodiments of the present technology.

FIG. 6 illustrates various shapes for the distal portion of the catheter of FIG. 1 in accordance with additional embodiments of the present technology.

FIGS. 7A-7C are side views of a distal portion of a clot treatment system in accordance with additional embodiments of the present technology.

### DETAILED DESCRIPTION

The present technology is generally directed to aspiration guide catheters having a pre-shaped distal portion for improved flexibility through tortuous/hard-to-reach vascular anatomy, and associated systems and methods. In some embodiments, an aspiration guide catheter includes an inner liner defining a lumen and having a proximal region and a distal region. The catheter can further include a braid of wires over the inner liner, and a wire coiled around the braid over at least a portion of the distal region of the inner liner. At least a portion of the braid over the distal region of the inner liner is configured to deflect away from a longitudinal axis of the proximal region to define a distal shaped portion of the catheter. In some embodiments, the distal shaped portion of the catheter can comprise a curved portion. The catheter can further include an outer sheath over the braid, the wire, and the inner liner and securing these components together.

In some aspects of the present technology, the catheter is configured to be steered to and positioned in difficult-to-reach regions of the anatomy (e.g., the venous anatomy) of a patient while still having a relatively large lumen (e.g., 20 French, 24 French, greater than 24 French). More particularly, the catheter can have an improved torque response and flexibility compared to conventional catheters having the same lumen size. For example, the braid can provide good torque response along the entire length of the catheter, while the shaped portion is configured to flex into and be positioned within the difficult-to-reach regions of the anatomy. In some embodiments, the outer sheath can have a hardness that decreases in the distal direction to provide (i) good pushability/torquability at the proximal region and (ii) good flexibility at the distal region. Further, the coil can be configured to provide a selected hoop strength at the distal region while still allowing the catheter to flex. For example, the coil can inhibit or even prevent kinking or other unwanted movement of the catheter when the lumen is aspirated during a clot removal procedure.

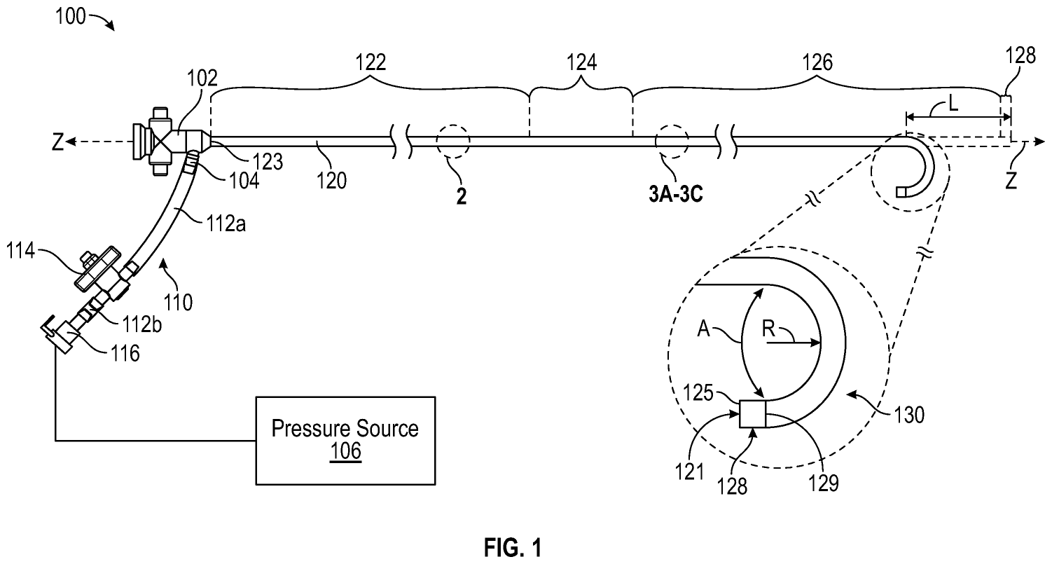

Certain details are set forth in the following description and in FIGS. 1-7C to provide a thorough understanding of various embodiments of the present technology. In other instances, well-known structures, materials, operations, and/or systems often associated with intravascular procedures, clot removal procedures, catheters, and the like are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

FIG. 1 is a partially schematic side view of a clot treatment system 100 in accordance with embodiments of the present technology. The clot treatment system 100 can also be referred to as an aspiration assembly, a clot removal system, and/or a thrombectomy system. In the illustrated embodiment, the clot treatment system 100 includes a tubing assembly 110 fluidly coupled to a catheter 120 via a valve 102. In general, the clot treatment system 100 (i) can include features generally similar or identical to those of the clot treatment systems described in detail in U.S. patent application Ser. No. 16/536,185, filed Aug. 8, 2019, and titled “SYSTEM FOR TREATING EMBOLISM AND ASSOCIATED DEVICES AND METHODS,” which is incorporated herein by reference in its entirety, and/or (ii) can be used to treat/remove clot material from a patient (e.g., a human patient) using any of the methods described in detail therein.

In the illustrated embodiment, the catheter 120 includes (i) a proximal region or portion 122, (ii) an intermediate region 124 adjacent to and distal of the proximal region 122, (iii) a distal region 126 adjacent to and distal of the intermediate region 124, and (iv) a distal tip region 128 adjacent to and distal of the distal region 126 (collectively “regions 122-128”). The catheter 120 further defines a lumen 121 extending entirely therethrough from the proximal region 122 to the distal tip region 128. The proximal region 122 defines a proximal terminus 123 of the catheter 120, and the distal tip region 128 defines a distal terminus 125 of the catheter 120. In the illustrated embodiment, the distal tip region 128 includes a marker band 129, such as a radiopaque marker configured to facilitate visualization of the position of the catheter 120 during a medical procedure (e.g., a clot removal procedure) using the catheter 120.

In some embodiments, the proximal region 122 has a first length, the intermediate region 124 has a second length less than the first length, the distal region 126 has a third length greater than the second length but less than the first length, and the distal tip region 128 has a fourth length less than the first, second, and third lengths. For example, the first length can be between about 20.0-28.0 inches (e.g., about 24.0 inches), the second length can be between about 2.0-3.0 inches (e.g, about 2.50 inches), the third length can be between about 11.00-15.00 inches (e.g., about 13.25 inches), and the fourth length can be between about 0.10-0.50 inch (e.g., about 0.25 inch). In other embodiments, the lengths of one or more of the regions 122-128 can be different.

In some embodiments, the catheter 120 can have varying flexibilities, shapes, thicknesses, and/or other properties in/along the various regions 122-128. For example, the distal region 126 can include a shaped portion 130 configured (e.g., heat set) to deflect away from a longitudinal axis Z of the catheter 120 relative to the rest of the distal region 126, the intermediate region 124, and the proximal region 122. That is, the shaped portion 130 can be configured to deflect away from the longitudinal axis Z that is aligned with the proximal region 122, the intermediate region 124, and/or the unshaped portion of the distal region 126 proximal to the shaped portion 130. In the illustrated embodiment, the shaped portion 130 has a generally curved shape. In some embodiments, the shaped portion 130 can move between (i) a relaxed position in which the shaped portion 130 has the curved shape illustrated in FIG. 1 and (ii) a constrained position in which the shaped portion 130 is more closely aligned with the longitudinal axis Z, as shown in phantom lines in FIG. 1. In some embodiments, the shaped portion 130 has (i) a length L that can be between about 1.0-5.0 inches (e.g., about 2.35 inches), (ii) a bend radius R of between about 0.5-1.0 inch (e.g., about 0.8 inch, less than about 0.8 inch), and/or (iii) a bend angle A of between about 165-195 degrees (e.g., about 180 degrees) or greater than 195 degrees (e.g., between about 250-290 degrees, about 270 degrees). In other embodiments, the bend angle A can be less than 165 degrees.

In other embodiments, the shaped portion 130 can have other dimensions and/or shapes. For example, FIG. 6 illustrates various shapes for the shaped portion 130 of the catheter 120 in accordance with additional embodiments of the present technology. The shaped portion 130 can have any of the shapes illustrated in FIG. 6, such as a Tiger curve shape, a Jacky curve shape, an Amplatz left shape, an LCB shape, an RCB shape, a Judkins left shape, a Judkins right shape, a Multipurpose A2 shape, an IM shape, a 3D LIMA shape, a IM VB-1 shape, and so on.

The valve 102 is fluidly coupled to the lumen 121 of the catheter 120 and can be integral with or coupled to the proximal region 122 of the catheter 120. In some embodiments, the valve 102 is a hemostasis valve that is configured to maintain hemostasis during a clot removal procedure by preventing fluid flow in the proximal direction through the valve 102 as various components such as delivery sheaths, pull members, guidewires, interventional devices, other aspiration catheters, and so on are inserted through the valve 102 to be delivered through the catheter 120 to a treatment site in a blood vessel. The valve 102 includes a branch or side port 104 configured to fluidly couple the lumen 121 of the catheter 120 to the tubing assembly 110. In some embodiments, the valve 102 can be a valve of the type disclosed in U.S. patent application Ser. No. 16/117,519, filed Aug. 30, 2018, and titled “HEMOSTASIS VALVES AND METHODS OF USE,” which is incorporated herein by reference in its entirety.

In the illustrated embodiment, the tubing assembly 110 fluidly couples the catheter 120 to a pressure source 106, such as a syringe. The tubing assembly 110 can include one or more tubing sections 112 (individually labeled as a first tubing section 112a and a second tubing section 112b), at least one fluid control device 114 (e.g., a valve), and at least one connector 116 (e.g., a Toomey tip connector) for fluidly coupling the tubing assembly 110 to the pressure source 106 and/or other suitable components. In some embodiments, the fluid control device 114 is a stopcock that is fluidly coupled to (i) the side port 104 of the valve 102 via the first tubing section 112a and (ii) the connector 116 via the second tubing section 112b. The fluid control device 114 is externally operable by a user to regulate the flow of fluid therethrough and, specifically, from the lumen 121 of the catheter 120 to the pressure source 106. In some embodiments, the connector 116 is a quick-release connector (e.g, a quick disconnect fitting) that enables rapid coupling/decoupling of the catheter 120 and the fluid control device 114 to/from the pressure source 106.

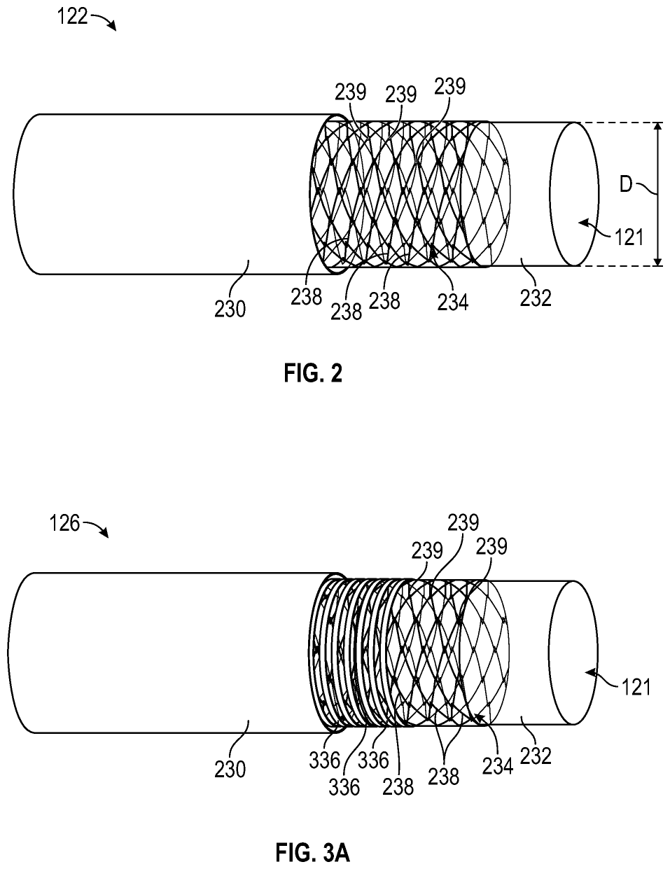

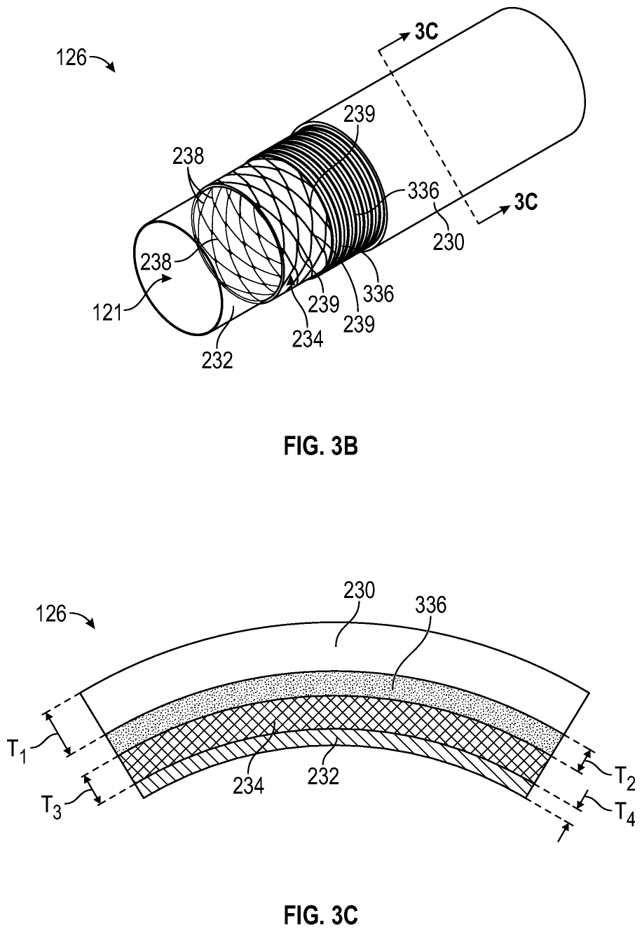

FIG. 2 is an enlarged, partial cut-away side view of a portion of the proximal region 122 of the catheter 120 in accordance with embodiments of the present technology. FIG. 3A is an enlarged, partial cut-away side view, and FIG. 3B is an enlarged, partial cut-away perspective view, of a portion of the distal region 126 of the catheter 120 in accordance with embodiments of the present technology. Referring to FIGS. 1-3B together, the catheter 120 includes an outer sheath 230 and an inner liner 232 extending through/defining each of the regions 122-128. The outer sheath 230 is positioned over (e.g., radially outside of) the inner liner 232. The outer sheath 230 can also be referred to as an outer jacket, an outer shaft, or an outer layer, and the inner liner 232 can also be referred to as an inner layer, an inner sheath, or an inner shaft.

The catheter 120 further includes (i) a braid 234 extending along each of the regions 122-128 between the outer sheath 230 and the inner liner 232 and (ii) a coil 336 (FIGS. 3A-3C) extending at least partially along the distal region 126 between the braid 234 and the outer sheath 230. In some embodiments, the coil 336 can extend from the distal region 126 at least partially (e.g., entirely) along the intermediate region 124 and/or the proximal region 122. In certain embodiments, the coil 336 is omitted in the proximal region 122 but extends substantially entirely along the intermediate region 124 and the distal region 126.

In some embodiments, the outer sheath 230 can be formed from a plastic material, elastomeric material, and/or thermoplastic elastomer (TPE) material. In some embodiments, the outer sheath 230 can be formed from a TPE manufactured by Arkema S.A., of Colombes, France, such as the TPEs manufactured under the trademark “Pebax.” In some embodiments, the outer sheath 230 can have a varying hardness (e.g., durometer), thickness, flexibility, rigidity, and/or other property in one or more of the different regions 122-128. For example, the outer sheath 230 can have a first hardness along the proximal region 122, a second hardness along the intermediate region 124 that is less than the first hardness, a third hardness along the distal region 126 that is less than the first hardness and the second hardness, and a fourth hardness in the distal tip region 128 that is greater than third hardness. In some embodiments, the first hardness and the fourth hardness can each be between about 65 D-75 D (e.g., about 72 D), the second hardness can be between about 50 D-60 D (e.g, about 55 D), and the third hardness can be between about 30 D-40 D (e.g., about 35 D). In other embodiments, one or more of the regions 122-128 can have a different hardness.

The inner liner 232 defines the lumen 121 and, in some embodiments, can be formed of a lubricious material that facilitates the movement (e.g., distal advancement, proximal retraction) of various components through the lumen 121, such as delivery sheaths, pull members, guidewires, interventional devices, other aspiration catheters, and the like. In some embodiments, the inner liner 232 can be formed from a polymer material, a fluoropolymer material (e.g., polytetrafluoroethylene (PTFE)), and/or another material having a high degree of lubricity. In some embodiments, the inner liner 232 can have a diameter D (FIG. 2) of greater than about 6 French, greater than about 10 French, greater than about 16 French, greater than about 20 French, greater than about 24 French, or greater. In some embodiments, the diameter D is about 8 French, about 16 French, about 20 French, or about 24 French. In certain embodiments, the diameter D of the inner liner 232 is the same in each of the regions 122-128 while, in other embodiments, the diameter D can vary along one or more of the regions 122-128.

The braid 234 can include wires, filaments, threads, sutures, fibers, or the like (collectively “wires 238”) that have been woven or otherwise coupled, attached, formed, and/or j oined together at a plurality of interstices 239. Accordingly, the braid 234 can also be referred to as a braided structure, a braided filament structure, a braided filament mesh structure, a mesh structure, a mesh filament structure, and the like. In some embodiments, the wires 238 can comprise metals, polymers, and/or composite materials. At least a portion of the wires 238 at the shaped portion 130 of the catheter 120 can be formed from a shape memory material, a shape memory alloy, and/or a shape memory polymer, such as nickel-titanium alloys (e.g., nitinol). In some embodiments, individual ones of the wires 238 can be rolled flat wires having a cross-sectional dimension of between about 0.001-0.005 inch (e.g., about 0.002 inch) by about 0.002-0.005 inch (e.g., about 0.0033 inch).

In the illustrated embodiment, the coil 336 is a single wire wound around the braid 234 and the inner liner 232. In other embodiments, the coil 336 can include more than one wire wound about the braid 234. For example, the coil 336 can include multiple wires wound over one another and/or multiple wires wound to at least partially overlap one another to form a braided or overlapping coil structure on the braid 234. In other embodiments, the coil 336 can be formed directly over the inner liner 232, and the braid 234 can be formed over the coil 336. The coil 336 can be formed from a metallic or other suitably strong material, such as nickel-titanium alloys (e.g. nitinol), platinum, cobalt-chrome alloys, stainless steel, tungsten, and/or titanium. In some embodiments, the coil 336 can include a shape memory material at least at the shaped portion 130 of the catheter 120.

FIG. 3C is an enlarged cross-sectional view of a portion of the distal region 126 of the catheter 120 taken along the line 3C-3C in FIG. 3B in accordance with embodiments of the present technology. In the illustrated embodiment, the outer sheath 230 has a first thickness T₁, the coil 336 has a second thickness T₂ less than the first thickness T₁, the braid 234 has a third thickness T₃ less than the first thickness T₁ but greater than the second thickness T₂, and the inner liner 232 has a fourth thickness T₄ less than the first, second, and third thicknesses T₁₋₃. For example, the first thickness T₁ can be between about 0.005-0.010 inch (e.g., between about 0.006-0.007 inch, about 0.006 inch, about 0.007 inch), the second thickness T₂ can be between about 0.001-0.005 inch (e.g., about 0.003 inch), the third thickness T₃ can be between about 0.002-0.006 inch (e.g., about 0.004 inch), and the fourth thickness T₄ can be between about 0.001-0.004 inch (e.g., about 0.002 inch). In other embodiments, one or more of the first through fourth thicknesses T₁₋₄ can be different.

In some embodiments, the catheter 120 can be formed about a mandrel or other elongate member. For example, the inner liner 232 can first be positioned about the mandrel. Then, the braid 234 can be formed (e.g., wound, braided) about the inner liner 232 around the mandrel. Next, the coil 336 can be wound around the mandrel about the braid 234 over the intermediate region 124 and the distal region 126 (e.g., of the inner liner 232 and the braid 234). In some embodiments, the marker band 129 can be positioned about the mandrel at the distal tip region 128. Next, the outer sheath 230 can be positioned over the inner liner 232, the braid 234, and the coil 336, and then heat shrunk or otherwise secured thereto. In some embodiments, the outer sheath 230 can be fused to the coil 336, the braid 234, and/or the inner liner 232 to secure these components of the catheter 120 together.

Finally, the shaped portion 130 of the catheter 120 can be shaped using a heat setting or other suitable process to have the curved shape illustrated in FIG. 1, or another shape (e.g., any of the shapes shown in FIG. 6). For example, as is known in the art of heat setting shape memory structures, a fixture, mandrel, or mold may be used to hold the shaped portion 130 in its desired shape, and then the shaped portion 130 can be subjected to an appropriate heat treatment such that the wires 238 of the braid 234 and/or the coil 336 assume or are otherwise shape-set to the outer contour of the mandrel or mold. In some embodiments, only the wires 238 of the braid 234 include a shape memory material that is shaped via the heat setting process. In other embodiments, the coil 336 can alternatively or additionally include a shape memory material that is shaped via the heat setting process. The heat setting process may be performed in an oven or fluidized bed, as is well-known. Therefore, the heat setting process can impart a desired shape, geometry, bend, and/or curve in the super-elastic and/or shape memory material or materials used to form the braid 234 and/or the coil 336. Accordingly, the shaped portion 130 may be radially constrained without plastic deformation (e.g., as shown in phantom in FIG. 1) and will self-expand on release of the radial constraint to the position illustrated in FIG. 1.

In some aspects of the present technology, the catheter 120 is configured to be steered to and positioned in difficult-to-reach regions of the anatomy (e.g., venous anatomy) of a patient while still having a relatively large size (e.g., 20 French, 24 French, greater than 24 French). More particularly, the catheter 120 can have an improved torque response and flexibility compared to conventional catheters having the same size. For example, the braid 234 can provide good torque response along the entire length of the catheter 120 because the braid 234 extends entirely through each of the regions 122-128 of the catheter 120. Moreover, the varying hardness (e.g., distally decreasing hardness) of the outer sheath 230 can provide (i) good torque response and/or pushability at the proximal region 122 and (ii) increased flexibility at the intermediate and distal regions 124, 126. Additionally, the shaped portion 130 is configured (e.g., shaped, sized, positioned) to flex into and be positioned within the difficult-to-reach regions of the anatomy. Further, the coil 336 can provide increased hoop strength at the distal region 126 while still allowing the catheter 120 to flex. For example, the coil 336 can inhibit or even prevent kinking or other unwanted movement of the catheter 120 when the lumen 121 is aspirated during a clot removal procedure.

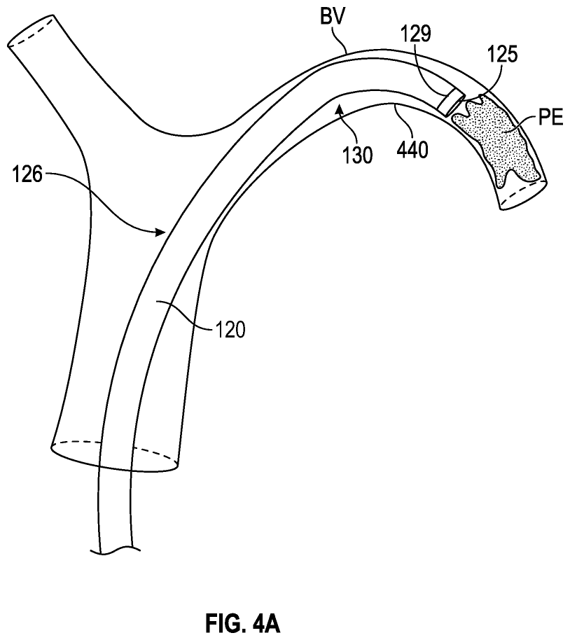

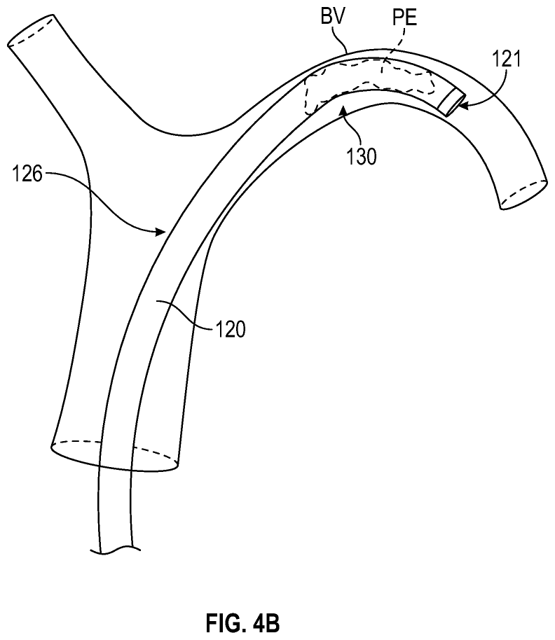

FIGS. 4A and 4B are side views of the distal region 126 of the catheter 120 of the clot treatment system 100 during a procedure for removing clot material PE (e.g., a pulmonary embolism) from within a blood vessel BV (e.g., a pulmonary blood vessel) of a patient (e.g., a human patient) in accordance with embodiments of the present technology. As noted above, in some embodiments the clot removal procedure illustrated in FIGS. 4A and 4B can be generally similar or identical to any of the clot removal procedures disclosed in U.S. patent application Ser. No. 16/536,185, filed Aug. 8, 2019, and titled “SYSTEM FOR TREATING EMBOLISM AND ASSOCIATED DEVICES AND METHODS,” which is incorporated herein by reference in its entirety.

With reference to FIGS. 1 and 4A together, the catheter 120 can be advanced through the patient to proximate the clot material PE with the blood vessel BV (e.g., advanced to a treatment site within the blood vessel BV). In some embodiments, the catheter 120 can be advanced through the blood vessel BV until the distal terminus 125 of the catheter 120 is positioned proximate to a proximal portion of the clot material PE. In some embodiments, the position of the distal terminus 125 can be confirmed or located via visualization of the marker band 129 using fluoroscopy or another imaging procedure (e.g., a radiographic procedure). In other embodiments, the distal terminus 125 can be positioned at least partially within the clot material PE or distal of the clot material PE.

In some aspects of the present technology, the shaped portion 130 helps the catheter 120 flex/bend into tortuous (e.g., hard-to-reach) regions of the blood vessel BV. For example, in the illustrated embodiment the shaped portion 130 has flexed around a bend 440 in the blood vessel BV that can have a relatively small radius of curvature. The portion of the blood vessel BV distal of the bend 440 can be difficult to reach due to the required approach angle, varying anatomy of the blood vessel BV, and/or irregularities due to illness of the patient. In some embodiments, the blood vessel BV can be a portion of left pulmonary artery, the temporal arteries, the inferior vena cava, or the right atrium. In some embodiments, the clot material PE can be a clot in transit (CIT) within the right atrium.

In some embodiments, before advancing the catheter 120 to the position shown in FIG. 4A, the catheter 120 can be rotated to align the shaped portion 130 with the bend 440 (e.g., to at least generally align a deflection direction of the shaped portion 130 with the bend 440). In contrast to the catheter 120 of the present technology, conventional catheters of the same size may be too stiff to easily position proximate the clot material PE. For example, such conventional catheters may “rainbow” over the clot material PE by following or tracking along the wall of the blood vessel BV at the outside of the bend 400. In addition to the shaped portion 130, both (i) the varying hardness of the outer sheath 230 (FIGS. 2-3C) and (ii) the flexibility of the braid 234 (FIGS. 2-3C) and the coil 446 (FIGS. 3A-3C) can help the catheter 120 flex through the anatomy of the blood vessel BV to the desired position proximate the clot material PE.

Access to the pulmonary vessels can be achieved through the patient's vasculature, for example, via the femoral vein. In some embodiments, the clot treatment system 100 can include an introducer (e.g., a Y-connector with a hemostasis valve; not shown) that can be partially inserted into the femoral vein. A guidewire (not shown) can be guided into the femoral vein through the introducer and navigated through the right atrium, the tricuspid valve, the right ventricle, the pulmonary valve, and into the main pulmonary artery. Depending on the location of the clot material PE, the guidewire can be guided to one or more of the branches of the right pulmonary artery and/or the left pulmonary artery. In some embodiments, the guidewire can be extended entirely or partially through the clot material PE. In other embodiments, the guidewire can be extended to a location just proximal of the clot material PE. After positioning the guidewire, the catheter 120 can be placed over the guidewire and advanced to the position proximate to the clot material PE as illustrated in FIG. 4A. In some embodiments, the guidewire can then be withdrawn while, in other embodiments, the guidewire can remain and can be used to guide other catheters (e.g., delivery catheters, additional aspiration guide catheters), interventional devices, etc., to the treatment site. It will be understood, however, that other access locations into the venous circulatory system of a patient are possible and consistent with the present technology. For example, the user can gain access through the jugular vein, the subclavian vein, the brachial vein, or any other vein that connects or eventually leads to the superior vena cava. Use of other vessels that are closer to the right atrium of the patient's heart can also be advantageous as it reduces the length of the instruments needed to reach the clot material PE.

With reference to FIGS. 1 and 4B together, the pressure source 106 is configured to generate (e.g., form, create, charge, build-up) a vacuum (e.g., negative relative pressure) and store the vacuum for subsequent application to the catheter 120. For example, after positioning the catheter 120 proximate the clot material PE, a user can first close the fluid control device 114 before generating the vacuum in the pressure source 106 by, for example, withdrawing the plunger of a syringe coupled to the connector 116. In this manner, a vacuum is charged within the pressure source 106 (e.g., a negative pressure is maintained) before the pressure source 106 is fluidly connected to the lumen 121 of the catheter 120. To aspirate the lumen 121 of the catheter 120, the user can open the fluid control device 114 to fluidly connect the pressure source 106 to the catheter 120 and thereby apply or release the vacuum stored in the pressure source 106 to the lumen 121 of the catheter 120.

Opening of the fluid control device 114 instantaneously or nearly instantaneously applies the stored vacuum pressure to the tubing assembly 110 and the catheter 120, thereby generating a suction pulse throughout the catheter 120. In particular, the suction is applied at the distal tip region 128 of the catheter 120 to suck/aspirate at least a portion of the clot material PE into the lumen 121 of the catheter 120, as shown in FIG. 4B. In one aspect of the present technology, pre-charging or storing the vacuum in the pressure source 106 before applying the vacuum to the lumen 121 of the catheter 120 is expected to generate greater suction forces and corresponding fluid flow velocities at and/or near the distal tip region 128 of the catheter 120 compared to simply activating the pressure source 106 while it is fluidly connected to the catheter 120.

Sometimes, as shown in FIG. 4B, discharging the vacuum stored in the pressure source to aspirate the lumen 121 of the catheter 120 may remove substantially all (e.g., a desired amount) of the clot material PE from the blood vessel BV. That is, a single aspiration pulse may adequately remove the clot material PE from the blood vessel BV. In other embodiments, a portion of the clot material PE may remain in the blood vessel BV. In such instances, the user may wish to again apply vacuum pressure (conduct an “aspiration pass”) to remove all or a portion of the remaining clot material PE in the blood vessel BV. In such instances, the pressure source 106 can be disconnected from the tubing assembly 110 and drained (e.g., aspirated clot removal removed) before the pressure source 106 is reconnected to the tubing assembly 110 and activated once again. After removing a desired amount of the clot material PE, the catheter 120 can be withdrawn from the patient.

In some aspects of the present technology, the relatively great flexibility and torquability of the catheter 120 allow the catheter 120 to be positioned in difficult-to-reach areas of the blood vessel BV (or elsewhere in the vasculature of the patient) without reducing the size of the lumen 121. It is expected that the increased size of the lumen 121 will provide greater suction forces over a smaller period of time (e.g., will provide a larger vacuum impulse). In some embodiments, the greater suction forces can facilitate the removal of clot material from a blood vessel of a patient even where the clot material is strongly lodged or attached within the blood vessel (e.g., a chronic clot). Accordingly, in contrast to conventional catheters, the catheter 120 can be used to generate greater aspirational forces for improved clot removal in hard-to-reach places of the vasculature. In additional aspects of the present technology, the coil 336 (FIGS. 3A-3C) can provide a high hoop strength along the distal region 126 of the catheter 120 that inhibits or even prevents kinking or other unwanted movement of the catheter 120 when the pressure source 106 is used to generate a suction pulse at the distal region 126 of the catheter 120.

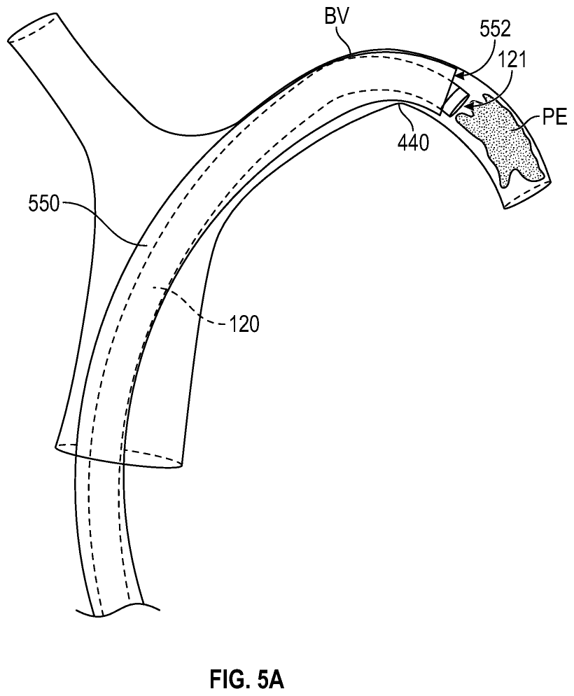

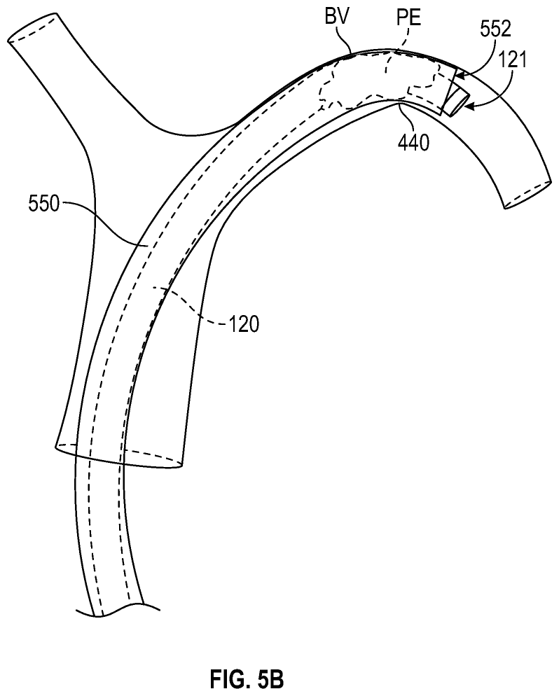

FIGS. 5A and 5B are side views of the distal region 126 of the catheter 120 of the clot treatment system 100 during another procedure for removing the clot material PE from within the blood vessel BV in accordance with additional embodiments of the present technology. As noted above, in some embodiments the clot removal procedure illustrated in FIGS. 5A and 5B can be generally similar or identical to any of the clot removal procedures disclosed in U.S. patent application Ser. No. 16/536,185, filed Aug. 8, 2019, and titled “SYSTEM FOR TREATING EMBOLISM AND ASSOCIATED DEVICES AND METHODS,” which is incorporated herein by reference in its entirety.

Referring first to FIG. 5A, the catheter 120 (“first catheter 120”) can be advanced through the patient to proximate the clot material PE with the blood vessel BV as described in detail above with reference to FIG. 4A. As shown in FIG. 5A, in some embodiments a second catheter 550 can then be advanced over the first catheter 120 to proximate the clot material PE. The second catheter 550 can define a lumen 552 having a larger size (e.g., 20 French or greater, 24 French or greater) than the lumen 121 of the first catheter 120. In some embodiments, the second catheter 550 has a greater stiffness than the first catheter 120. In other embodiments, the second catheter 550 can be larger than but generally similar or identical to the first catheter 120 (e.g., having a distal heat-set shaped portion).

Accordingly, the first catheter 120 can act as a guide or rail for guiding the advancement of the second catheter 550 to proximate the clot material PE. In some aspects of the present technology, the larger size and/or stiffness of the second catheter 550 could make the second catheter 550 difficult to navigate to proximate the clot material PE (e.g., across the bend 440) without the first catheter 120 acting as a guide. That is, the first catheter 120 can be flexibly positioned proximate the clot material PE and used to guide the larger and/or stiffer second catheter 550 into a position it would otherwise be difficult or impossible to do so.

The second catheter 550 can be part of a clot treatment system (not shown) having features generally similar or identical to that of the clot treatment system 100 of FIG. 1 including for example, a fluid control device and a pressure source. Accordingly, with reference to FIG. 5B, the pressure source can be used to generate a vacuum in the lumen 552 of the second catheter, and the fluid control device can be opened to fluidly connect the pressure source to the second catheter 550 and thereby apply or release the vacuum stored in the pressure source to the lumen 552 of the second catheter 550. Opening of the fluid control device instantaneously or nearly instantaneously applies the stored vacuum pressure to the second catheter 550, thereby generating a suction pulse throughout the second catheter 550 that can suck at least a portion of the clot material PE into the lumen 552 of the second catheter 550. In some embodiments, the clot treatment system 100 (FIG. 1) can also be operated to aspirate the clot material PE into the lumen 121 of the lumen 121 of the first catheter 120, as described in detail above with reference to FIGS. 4A and 4B. In some aspects of the present technology, the larger size of the lumen 552 of the second catheter 550 can be used to generate greater suction forces than the smaller lumen 121 of the first catheter 120. After removing a desired amount of the clot material PE, the first and second catheters 120, 550 can be withdrawn from the patient.

Referring to FIGS. 4A-5B together, in some embodiments one or more additional devices can be advanced through the catheter 120. For example, one or more aspiration catheters, mechanical thrombectomy devices, and/or the like can be advanced through the catheter 120 to proximate the clot material PE to aid in the clot removal process. In some such embodiments, the shaped portion 130 can facilitate the advancement of the one or more additional devices through the catheter 120. For example, the one or more additional devices can track through the shaped portion 130 of the catheter 120 to at and/or proximate the clot material PE.

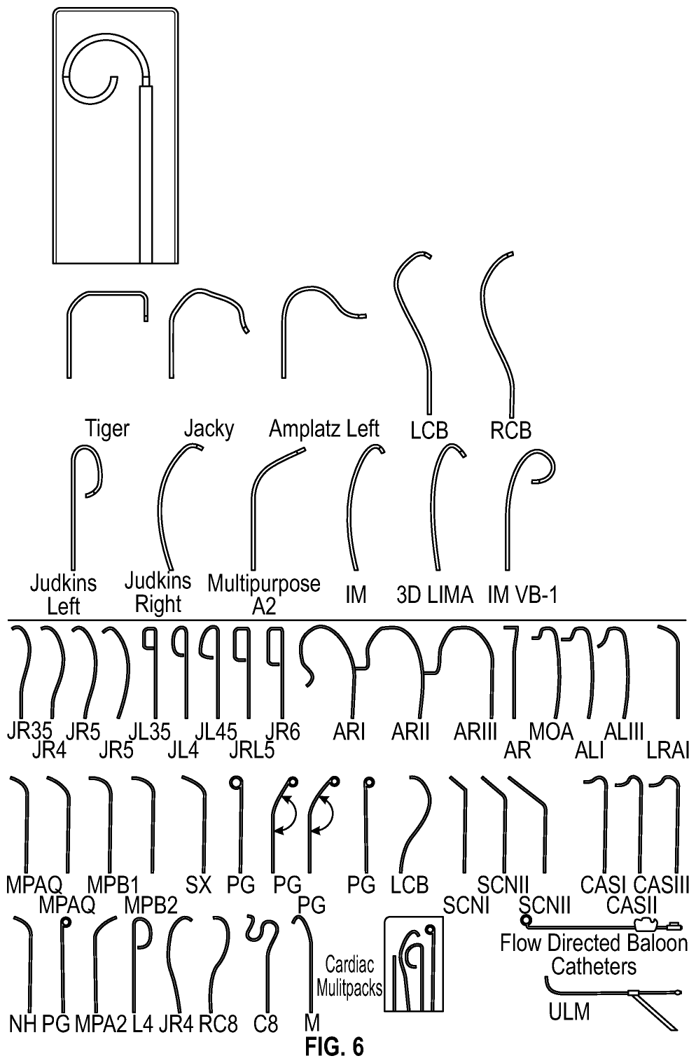

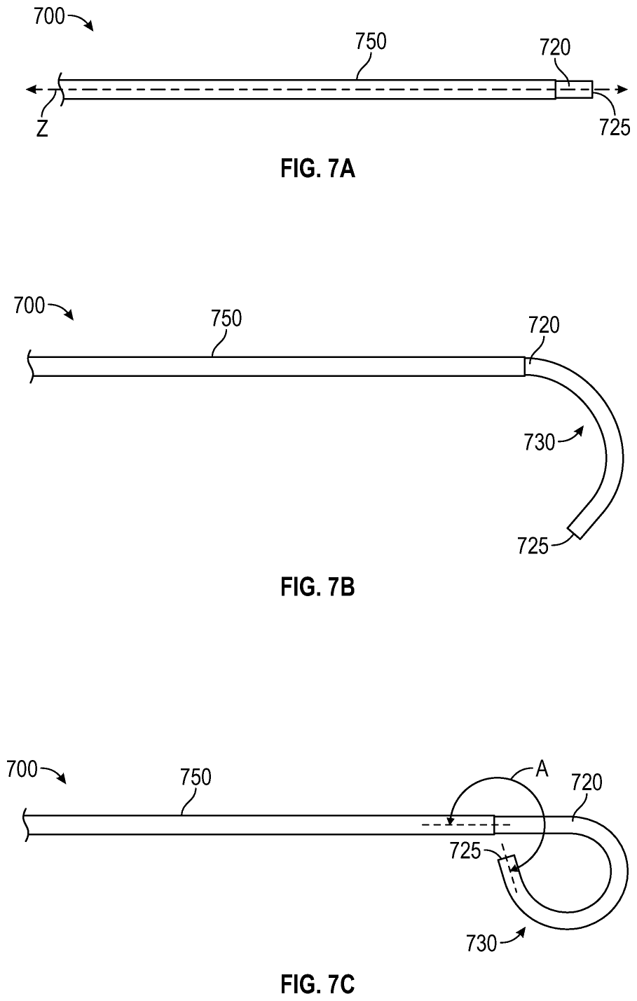

FIGS. 7A-7C are side views of a distal portion of a clot treatment system 700 in accordance with embodiments of the present technology. Referring to FIGS. 7A-7C together, the clot treatment system 700 can include some features that are least at generally similar in structure and function, or identical in structure and function, to the corresponding features of the clot treatment system 100 described in detail above with reference to FIGS. 1-6, and can operate in a generally similar or identical manner to the clot treatment system 100. In the illustrated embodiment, for example, the clot treatment system 700 includes a first catheter 720 (e.g., a pre-shaped catheter) that can be advanced through a larger second catheter 750. The first catheter 720 can have a shaped distal portion 730 (obscured in FIG. 7A) including a distal tip or terminus 725. In some embodiments the first catheter 720 has a size of about 20 French or greater than 20 French, and the second catheter 750 has a size of about 24 French or greater than 24 French.

FIGS. 7A-7C illustrate the sequential deployment of the shaped distal portion 730 from within the second catheter 750. More specifically, the shaped distal portion 730 is (i) positioned substantially within and constrained by the second catheter 750 in FIG. 7A, (ii) positioned partially within and partially outside the second catheter 750 in FIG. 7B, and (iii) positioned substantially outside of (e.g., distal of) and constrained by the second catheter 750 in FIG. 7C. In the illustrated embodiment, the shaped distal portion 730 is configured (e.g., heat set) to deflect away from a longitudinal axis Z (FIG. 7A) of the first and second catheters 720, 750 to have a generally curved shape. In some embodiments, the shaped distal portion 730 can deflect by an angle A of between about between about 250-290 degrees (e.g., about 255 degrees, about 260 degrees, about 270 degrees) when fully unconstrained by the second catheter 750 as shown in FIG. 7C. The shaped distal portion 730 can be deployed from the second catheter 750 via advancement of the first catheter 720 relative to the second catheter 750 and/or retraction of the second catheter 750 relative to the first catheter 720. That is, for example, the second catheter 750 can be retracted off of the shaped distal portion 730 such that the shaped distal portion 730 is no longer constrained by the second catheter 750 and thereby able to deflect away from the longitudinal axis Z as shown in FIG. 7C.

Several aspects of the present technology are set forth in the following examples:

1. An aspiration catheter, comprising:
an inner liner defining a lumen and having a proximal region and a distal region;
a braid of wires over the inner liner, wherein a deflectable portion of the braid over at least a portion of the distal region of the inner liner is configured to deflect away from a longitudinal axis of the proximal region when the aspiration catheter is unconstrained;
a wire coiled over at least a portion of the distal region of the inner liner; and
an outer sheath over the braid, the wire, and the inner liner.

2. The aspiration catheter of example 1 wherein the wires in the deflectable portion of the braid is pre-shaped to deflect away from the longitudinal axis.

3. The aspiration catheter of example 1 or example 2 wherein the deflectable portion of the braid is configured to deflect away from the longitudinal axis to have a bend angle of between about between about 165-195 degrees when the aspiration catheter is unconstrained.

4. The aspiration catheter of example 1 or example 2 wherein the deflectable portion of the braid is configured to deflect away from the longitudinal axis to have a bend angle of between about 270 degrees when the aspiration catheter is unconstrained.

5. The aspiration catheter of any one of examples 1-4 wherein the lumen has a diameter of 20 French or greater.

6. The aspiration catheter of any one of examples 1-5 wherein the lumen has a diameter of 24 French or greater.

7. The aspiration catheter of any one of examples 1-6 wherein the inner liner is formed from polytetrafluoroethylene material, and wherein the outer sheath is formed from a thermoplastic elastomer material.

8. The aspiration catheter of any one of examples 1-7 wherein the outer sheath has a first hardness over the proximal region of the inner liner, and wherein the outer sheath has a second hardness over the distal region of the inner liner that is less than the first hardness.

9. An aspiration catheter, comprising:
a proximal region defining a longitudinal axis;
an intermediate region extending from the proximal region;
a distal region extending from the intermediate region;
an inner lining defining a lumen and extending through the proximal region, the intermediate region, and the distal region;
a braid of wires extending over the inner liner and through the proximal region, the intermediate region, and the distal region, wherein a deflectable portion of the braid in the distal region is configured to deflect away from the longitudinal axis when the aspiration catheter is unconstrained;
a wire coiled in the distal region; and
an outer sheath extending over the braid, the wire, and the inner liner and through the proximal region, the intermediate region, and the distal region.

10. The aspiration catheter of example 9 wherein the deflectable portion of the braid is configured to deflect away from the longitudinal axis to have a bend angle of between about between about 165-195 degrees when the aspiration catheter is unconstrained.

11. The aspiration catheter of example 9 or example 10 wherein the outer sheath has (a) a first hardness in the proximal region of between about 65 D-75 D, (b) a second hardness in the intermediate region of between about 50 D-60 D, and (c) a third hardness in the distal region of between about 30 D-40 D.

12. The aspiration catheter of any one of examples 9-11 wherein the outer sheath has (a) a first hardness in the proximal region, (b) a second hardness in the intermediate region less than the first hardness, and (c) a third hardness in the distal region less than the second hardness.

13. The aspiration catheter of example 12 wherein (a) the proximal region has a first length, (b) the intermediate region has a second length less than the first length, and (c) the distal region has a third length greater than the second length and less than the first length.

14. The aspiration catheter of example 13 wherein the first length is between about 20.0-28.0 inches, the second length is between about 2.0-3.0 inches, and the third length is between about 11.00-15.00 inches.

15. A method of removing clot material from a blood vessel, the method comprising:
advancing an aspiration catheter through the blood vessel, wherein the aspiration catheter includes a distal portion and a proximal portion, wherein the distal portion is configured to deflect away from a longitudinal axis of the proximal portion;
positioning a distal tip of the aspiration catheter proximate the clot material;
activating a pressure source coupled to the aspiration catheter via a fluid control device, while the fluid control device is closed, to generate a vacuum in the pressure source; and
opening the fluid control device to apply the vacuum to the aspiration catheter to thereby aspirate at least a portion of the clot material into the aspiration catheter.

16. The method of example 15 wherein the distal portion of the aspiration catheter is configured to deflect away from the longitudinal axis in a deflection direction, and wherein the method further comprises rotating the aspiration catheter such that the deflection direction is at least partially aligned with a bend in the blood vessel.

17. The method of example 15 or example 16 wherein the aspiration catheter has a size of 20 French or greater.

18. The method of any one of examples 15-17 wherein the distal portion of the aspiration catheter is configured to deflect away from the longitudinal axis to have a bend angle of between about between about 165-195 degrees when the aspiration catheter is unconstrained.

19. The method of any one of examples 15-17 wherein the distal portion of the aspiration catheter is configured to deflect away from the longitudinal axis to have a bend angle of about 270 degrees when the aspiration catheter is unconstrained.

20. The method of any one of examples 15-19 wherein the distal portion of the aspiration catheter includes—
an inner liner defining a lumen;
a braid of wires extending over the inner liner;
a wire coiled around the braid; and
an outer sheath extending over the braid.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An aspiration catheter, comprising:
an inner liner defining a lumen and having a proximal region and a distal region;
a braid of wires over the inner liner, wherein a deflectable portion of the braid over at least a portion of the distal region of the inner liner is configured to deflect away from a longitudinal axis of the proximal region when the aspiration catheter is unconstrained;
a wire coiled over at least a portion of the distal region of the inner liner; and
an outer sheath over the braid, the wire, and the inner liner.

2. The aspiration catheter of claim 1 wherein the wires in the deflectable portion of the braid is pre-shaped to deflect away from the longitudinal axis.

3. The aspiration catheter of claim 1 wherein the deflectable portion of the braid is configured to deflect away from the longitudinal axis to have a bend angle of between about between about 165-195 degrees when the aspiration catheter is unconstrained.

4. The aspiration catheter of claim 1 wherein the deflectable portion of the braid is configured to deflect away from the longitudinal axis to have a bend angle of between about 270 degrees when the aspiration catheter is unconstrained.

5. The aspiration catheter of claim 1 wherein the lumen has a diameter of 20 French or greater.

6. The aspiration catheter of claim 1 wherein the lumen has a diameter of 24 French or greater.

7. The aspiration catheter of claim 1 wherein the inner liner is formed from polytetrafluoroethylene material, and wherein the outer sheath is formed from a thermoplastic elastomer material.

8. The aspiration catheter of claim 1 wherein the outer sheath has a first hardness over the proximal region of the inner liner, and wherein the outer sheath has a second hardness over the distal region of the inner liner that is less than the first hardness.

9. An aspiration catheter, comprising:
a proximal region defining a longitudinal axis;
an intermediate region extending from the proximal region;
a distal region extending from the intermediate region;
an inner lining defining a lumen and extending through the proximal region, the intermediate region, and the distal region;
a braid of wires extending over the inner liner and through the proximal region, the intermediate region, and the distal region, wherein a deflectable portion of the braid in the distal region is configured to deflect away from the longitudinal axis when the aspiration catheter is unconstrained;
a wire coiled in the distal region; and
an outer sheath extending over the braid, the wire, and the inner liner and through the proximal region, the intermediate region, and the distal region.

10. The aspiration catheter of claim 9 wherein the deflectable portion of the braid is configured to deflect away from the longitudinal axis to have a bend angle of between about between about 165-195 degrees when the aspiration catheter is unconstrained.

11. The aspiration catheter of claim 9 wherein the outer sheath has (a) a first hardness in the proximal region of between about 65 D-75 D, (b) a second hardness in the intermediate region of between about 50 D-60 D, and (c) a third hardness in the distal region of between about 30 D-40 D.

12. The aspiration catheter of claim 9 wherein the outer sheath has (a) a first hardness in the proximal region, (b) a second hardness in the intermediate region less than the first hardness, and (c) a third hardness in the distal region less than the second hardness.

13. The aspiration catheter of claim 12 wherein (a) the proximal region has a first length, (b) the intermediate region has a second length less than the first length, and (c) the distal region has a third length greater than the second length and less than the first length.

14. The aspiration catheter of claim 13 wherein the first length is between about 20.0-28.0 inches, the second length is between about 2.0-3.0 inches, and the third length is between about 11.00-15.00 inches.

15. A method of removing clot material from a blood vessel, the method comprising:
advancing an aspiration catheter through the blood vessel, wherein the aspiration catheter includes a distal portion and a proximal portion, wherein the distal portion is configured to deflect away from a longitudinal axis of the proximal portion;
positioning a distal tip of the aspiration catheter proximate the clot material;
activating a pressure source coupled to the aspiration catheter via a fluid control device, while the fluid control device is closed, to generate a vacuum in the pressure source; and
opening the fluid control device to apply the vacuum to the aspiration catheter to thereby aspirate at least a portion of the clot material into the aspiration catheter.

16. The method of claim 15 wherein the distal portion of the aspiration catheter is configured to deflect away from the longitudinal axis in a deflection direction, and wherein the method further comprises rotating the aspiration catheter such that the deflection direction is at least partially aligned with a bend in the blood vessel.

17. The method of claim 15 wherein the aspiration catheter has a size of 20 French or greater.

18. The method of claim 15 wherein the distal portion of the aspiration catheter is configured to deflect away from the longitudinal axis to have a bend angle of between about between about 165-195 degrees when the aspiration catheter is unconstrained.

19. The method of claim 15 wherein the distal portion of the aspiration catheter is configured to deflect away from the longitudinal axis to have a bend angle of about 270 degrees when the aspiration catheter is unconstrained.

20. The method of claim 15 wherein the distal portion of the aspiration catheter includes—
an inner liner defining a lumen;
a braid of wires extending over the inner liner;
a wire coiled around the braid; and
an outer sheath extending over the braid.
